(19) United States
(12) Patent Application Publication
(10) Pub. No.: US 2005/0209504 A1
(43) Pub. Date: Sep. 22, 2005

(54) Method of presenting audible and visual cues for synchronizing the breathing cycle with an external timing reference for purposes of synchronizing the heart rate variability cycle with the breathing cycle

(76) Inventor: Stephen Bennett Elliott, Allen, TX (US)

Correspondence Address: Stephen Bennett Elliott, 702 Buffalo Springs Drive, Allen, TX, 75013, US

(21) Appl. No.: 11/060,671

(22) Filed: Feb. 18, 2005

Related U.S. Application Data
(63) Continuation-in-part of application No. 10/802,456, filed on Mar. 18, 2004.

(51) Int. Cl. A61M 21/00; A61B 5/04; A61B 5/08

(52) U.S. Cl. 600/26; 128/905; 600/529

(57) ABSTRACT

The invention consists of a broad method of presenting audible, visual, and sensory cues for synchronizing the breathing cycle with an external timing reference for purposes of synchronizing the heart rate variability cycle with the breathing cycle, thereby achieving coherence of the heart rate variability cycle. A family of audible, visual, and sensory indicators is specified for purposes of communicating breathing phase, change of breathing phase, progression of time within a phase, and progression of the phase relative to the internal perception of the practitioner.

### RELATED PATENT FILINGS

Method and System for Consciously Synchronizing the Breathing Cycle with the Natural Heart Rate Cycle (Ser. No. 10/699,025), Method and System for Synchronizing the Heart Rate Variability Cycle With The Breathing Cycle (Feb. 19, 2004). This application is a Continuation In Part of prior application Ser. No. 10/802,456 Method of Presenting Audible and Visual Cues for Synchronizing the Breathing Cycle With An External Timing Reference for Purposes of Synchronizing the Heart Rate Variability Cycle With The Breathing Cycle.

### FIELD OF THE INVENTION

The present invention relates to human physiology, and in particular to a method and system for allowing a human subject to consciously control physiological processes, more particularly, it allows a human subject to achieve synchronization of the natural cycle of heart rate with the breathing cycle.

### BACKGROUND OF THE INVENTION

The human heart is known to have its own nervous system and its own natural tendency toward rhythm. For purposes of this invention, there are two primary aspects to this rhythm, the heartbeat rate, and the rate at which the heartbeat rate changes otherwise known as heart rate variability. Heartbeat rate is usually specified in absolute number of heartbeats occurring during a specified period. Heartbeat rate variability, otherwise know as heart rate variability is the change in heartbeat rate as occurs during a specified period. Henceforth, heartbeat rate variability will be referred to as heart rate variability.

While the heart has its own tendency toward rhythm, it is closely coupled to breathing. The relationship is such that as inhalation occurs, the heartbeat rate tends to increase and as exhalation occurs, the heartbeat rate tends to decrease. It is important to note that while the heartbeat rate and breathing rate influence each other, the relationship is a plesiochronous one, that is, they are independent rhythms that strongly influence but do not directly control each other.

It is generally recognized that heart rate variability is an indicator of physiological and emotional state, that is, irregular incoherent heart rate variability indicates a condition of physiological/psychological stress. Alternatively, a highly regular coherent heart rate variability is indicative of a condition of physiological/psychological harmony.

Accordingly, it is highly desirable to achieve and maintain a highly coherent heart rate variability as life circumstances permit. This having been said, with proper training and the application of the present invention, it is possible for a human subject to rapidly achieve the desired state of high coherence of heart rate variability and to reinforce that coherence on an ongoing basis.

The present invention takes advantage of the relationship between the breathing cycle and the natural heart rate variability cycle to bring heart rate variability to the desired state of coherence and the human subject to the resultant state of physiological and emotional harmony. It accomplishes this via synchronization of the heart rate variability cycle with the breathing cycle. More specifically, it accomplishes this by providing audible and visual and sensory cues of varying formats which provide the practitioner with an accurate representation of a timing reference signal to which the breathing cycle is consciously synchronized.

### SUMMARY OF THE INVENTION

As previously described, a relationship exists between the heartbeat rate specified in terms of heart rate variability, and the breathing cycle. While the heart has its own tendency toward a natural variable rhythm, there is a strong correlation with breathing according to this specific relationship: as inhalation occurs, there is a tendency for the heartbeat rate to increase, as exhalation occurs, there is a tendency for the heartbeat rate to decrease. In a relaxed or semi-active human subject, the effect of the breathing cycle on the heart rate variability cycle is extremely strong. In fact, the heart rate variability cycle will synchronize with the breathing cycle if the breathing cycle is highly attuned to the periodicity of the natural heart rate variability cycle. The nominal period of the typical human heart rate variability cycle is 11.76 seconds. Therefore, if the period of the breathing cycle is timed to 11.76 seconds, the heart rate variability cycle will synchronize with it, bringing the natural heart rate variability cycle into phase synchrony with the breathing cycle and thereby bringing the subject's heart rate variability cycle into the desired state of coherence.

The present invention accomplishes this by presenting the human subject with various forms of audible and visual and sensory cues individually or in combination to which the breathing cycle is consciously synchronized. These audible and visual and sensory cues are synchronous signals with a nominal periodicity of 11.76 seconds divided by 2, or 5.88 seconds, representing the 50% of the typical 11.76 second heart rate variability cycle and corresponding to 50% of the breathing cycle of like period, that is, the period of inhalation or exhalation. When the breathing is consciously synchronized to this external timing reference signal, the heart rate variability cycle will synchronize with it and remain synchronized as long as the breathing cycle remains aligned with the tuned external reference. In this way, the human subject can remain in the desired state of coherence of heart rate variability for extended periods of time. Ultimately, this builds familiarity with the desired psycho-physiological condition such that synchronization with the external reference occurs subliminally and with continued practice, the state can be realized at will with or without the external timing reference signal.

For purposes of the present invention, we can consider the cycles of heart rate variability, the periodicity of increasing and decreasing of heartbeat rate, and the breathing cycle, the periodicity of inhalation and exhalation, to be two independent cycles. The relative synchronization of these cycles can vary between 0 and 180 degrees. When these cycles are completely out of phase, heart rate variability is maximally incoherent, when these cycles are completely in phase heart rate variability is maximally coherent.

Information pertaining to the interval can be simple or complex potentially including these information elements:
1) The moment of change from inhalation to exhalation
2) The moment of change from exhalation to inhalation
3) The phase of the breathing cycle, i.e. inhalation or exhalation.
4) The progression of the breathing cycle within a phase.

Owing to the fact that human sensory mechanisms are limited to audible, visual, and sensory forms of input, the number of ways that information pertaining to the period of an external reference signal generator can be conveyed is finite. This patent identifies all of the essential methods that are of practical concern. These methods can be generalized into audible, visual, and sensory forms per categories below:

Audible Methods:
1. The recurring recitation of a meaningful word, for example “inhale” or “exhale”,
2. The recurring recitation of numbers indicating phase and progression,
3. A recurring recognizable sound, for example a chime,
4. A recurring tempo or rhythm, for example, the strike of a bass drum,
5. A recurring change in volume (amplitude),
6. A recurring change in pitch (frequency),
7. A recurring melody or sequence,
8. A recurring change of ears, or stereo channels, and
9. Recurring change in psychoacoustic properties including:
   a. Perceived location in space, for example in front or behind,
   b. Perceived motion toward or away (Doppler effect), and

Visual Methods:
1. The recurring display of a meaningful word, for example “inhale” or “exhale,
2. The recurring display of numbers indicating phase and progression,
3. Recurring appearance and disappearance of an object,
4. Recurring visual motion, for example a point moving up and down or left and right,
5. Recurring increase and decrease in the size of an object,
6. Recurring change in color, including color attributes of hue, saturation, and luminance,
7. Recurring increase or decrease in rate of motion, for example a point or object that mimics vibration.

Sensory Methods:
1. The recurring simple vibratory pulse against the skin,
2. The recurring increase and decrease in vibration,
3. The recurring increase and decrease in pressure, for example by a pressure cuff, and
4. The recurring movement of a body part, for example the raising and lowering of a finger.

### BRIEF DESCRIPTION OF THE DRAWING FIGURES

The accompanying drawing figures incorporated in and forming a part of this specification illustrate several aspects of the invention, and together with the description serve to explain the principles of the invention.

FIG. 1 depicts the fundamental periodicity and logical representations of audible and visual indicators of varying utility.

FIG. 2 depicts various methods of presenting audible information representing the synchronized breathing cycle.

FIG. 3 presents an example of music with a tempo and audible cues specifically designed to align the breathing with a period of 5.9 seconds.

FIG. 4 presents an example of “mantra”, a word or phrase that is repeated over and over, again designed to align the breathing with a period of 5.9 seconds.

FIG. 5 presents the most basic binary representation of the objective breathing cycle.

FIG. 6 presents a vertically oriented 13 segment visual display.

FIG. 7 presents a vertically oriented oval visual display with 12 points.

FIG. 8 presents a 7 sector circular visual display

FIG. 9 presents additional audible indicator methods not specified in Ser. No. 10/802,456.

FIG. 10 presents additional visual indicator methods not specified in Ser. No. 10/802,456.

FIG. 11 presents sensory methods not specified in Ser. No. 10/802,456.

FIG. 12 presents nine segment visual display not specified in Ser. No. 10/802,456.

FIG. 13 presents a nine segment circular visual display not specified in Ser. No. 10/802,456.

FIG. 14 presents a nine segment polygonal display not specified in Ser. No. 10/802,456.

### DETAILED DESCRIPTION OF THE PREFERRED EMBODIMENTS

The present invention describes a series of related audible and visual methods by which information relating to the periodicity of an external timing reference is conveyed to a human practitioner for purposes of synchronizing their breathing cycle with the external reference signal for purposes of synchronizing their heart rate variability cycle with their breathing cycle. It is understood that these methods may ultimately be instantiated in any number of devices employing numerous forms of media including audio tape, compact disk, digital video disk, computers, hardware or software synthesizers, telephones, cellular telephones, televisions or radio broadcast, clocks, watches, and purpose built specialty devices. Secondly, audible forms of the invention may be instantiated in any or all forms of audible media including but not limited to music, background ambience generators, clocks of varying variety such as grandfather clocks, alarm clocks, telephones, and audible network based services such as radio stations, internet based services, and purpose built specialty devices. Thirdly, it is understood that there are an infinite number of variations possible relative to how an external timing reference may be communicated to a human participant or participants for the purpose of consciously synchronizing the participant's breathing with the external timing reference. The embodiments set forth below represent the necessary information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

The present invention allows a human subject to achieve coherence of heart rate variability by synchronizing the heart rate variability cycle with the breathing cycle. This is accomplished by providing an external timing reference in the form of an audible or visual signal, indicating when the subject should begin inhalation and when the subject should begin exhalation. In some cases, more complete information is provided indicating when the subject should begin inhalation, when the subject should end inhalation, when the subject should begin exhalation, and when the subject should end exhalation. This is repeated in a cyclic fashion, inhalation leading to exhalation, exhalation leading to inhalation, and so forth. The audible or visual reference signal is centered around the nominal frequency of 0.085 Hertz for a period of approximately 11.8 seconds, the center heart rate variability period of the typical human in a resting or semi-active state. When the typical human subject breathes at this rate, the heart rate variability cycle will synchronize with the breathing cycle, thereby maximizing the coherence of the heart rate variability cycle.

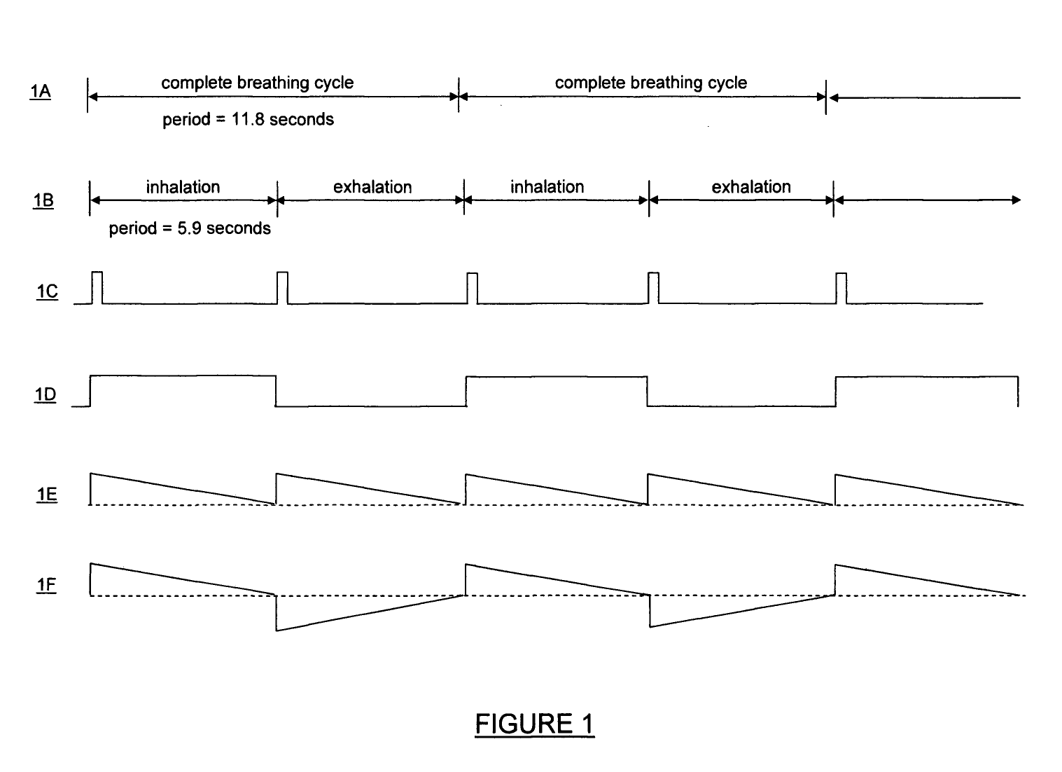

FIG. 1 presents basic timing and audible and visual functions in a logical format. The most basic information that must be conveyed to a subject or subjects practicing the method is the basic periodicity of the nominal 11.8 second breathing cycle 1A, divided by 2, yielding a 5.9 second period of inhalation and a 5.9 second period of exhalation 1B. This most basic indication is conveyed in the form of an audible or visual indicator of short duration 1C indicating when to change from inhalation to exhalation or when to change from exhalation to inhalation. 1D conveys this information as a change of phase, that is, a change from inhalation to exhalation or the reverse. Because very exacting coordination of the breath is required, a higher order signal indicating the moment of change as well as the progression of time is desirable 1E. Yet a further improvement that conveys change, progression, and distinguishes between inhalation and exhalation phases of activity is depicted by 1F.

To maximize heart rate variability coherence, it is extremely important that the timing of transitions from inhalation to exhalation and exhalation to inhalation be very exact. For this reason, it is highly desirable to provide the practitioner with audible or visual information such that they can understand breathing progression within a cycle, that is, within the 5.9 second period. By providing information relating to both progression and changes, the practitioner is much more able to align their breathing cycle with the external reference signal in an exacting fashion.

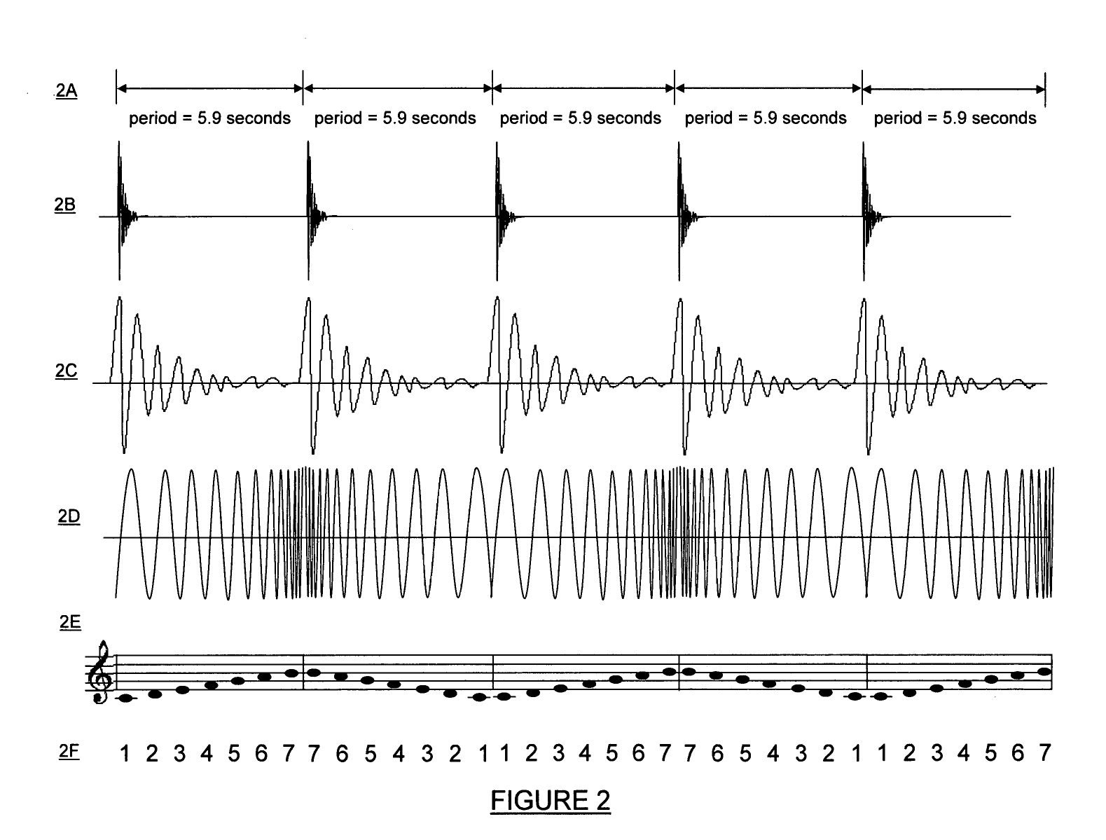

With reference to FIG. 2, five basic methods for communicating audible information are exemplified. Each method will now be explained. 2A depicts the basic 5.9 second period to which the breath in aligned. 2B is the most basic case representing a tone, chime, or tick conveying information pertaining to the change of the breathing cycle, that is, with each tone, chime, or tick, the breathing cycle changes from inhalation to exhalation or from exhalation to inhalation.

2C depicts the case where information pertaining to both change and progression is provided. This case might be typified by plucking the string of a stringed instrument such as a guitar wherein there is a rapid attack followed by a gradual decay, the rapid attack representing the moment of change of the breathing cycle and the gradual decay representing the progression of the 5.9 second interval. In this exemplary embodiment, the rapid attack occurs every 5.9 seconds. The decay occurs across the 5.9 second interval such that the audio amplitude becomes zero just prior to the next attack, indicating to the practitioner that a change in breathing cycle is imminent.

2D employs frequency modulation as the basis of indicating changes and progression. In this case, an audible tone is varied in frequency, the highest and lowest frequencies indicating the moment of the change in the breathing cycle from inhalation to exhalation or from exhalation to inhalation, respectively. This method has the advantage of facilitating inhalation with increasing frequency and exhalation with decreasing frequency, a convention that is useful both for audible-only cuing and for combined audible and visual cuing as will be explained later.

2E employs a similar method to that of 2D but using discrete musical notes as opposed as a continuously variable tone. In this example, changes in breathing cycle occur in between the two highest and two lowest notes. In this example, 7 notes are employed to represent the 5.9 second period. It should be noted that 7 notes represents a preferred embodiment of the present invention and that the application of any number of notes within a 5.9 second period provided for purposes of synchronizing breathing either consciously or subconsciously is intended within the scope of this disclosure. 20E provides the same advantage offered by 20D, that being the facilitation of inhalation with increasing frequency and exhalation with decreasing frequency. The seven successive notes C, D, E, F, G, A, and B occurring during the 5.9 second period is intentional, the number 7 being used consistently between both audible and visual presentations. Each note is sustained for a period of approximately 0.84 seconds.

FIG. 2F employs the simple method of counting, that is, the recitation of numbers 1 through 7 and 7 through 1. In this example, as in 2E, changes in breathing cycle occur in between the two highest numbers and two lowest numbers. Again this case offers the advantage of facilitating inhalation with increasing number and exhalation with decreasing number.

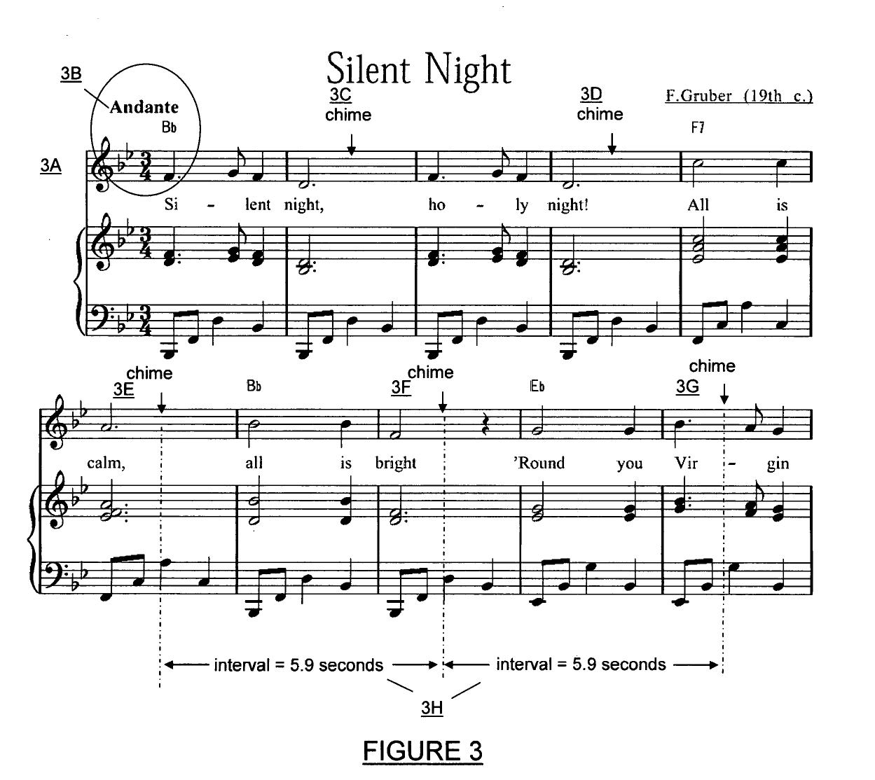

Referring now to FIG. 3, an essential embodiment of the present invention involves the adjustment of musical tempo and integration of audible cues into musical scores for the purpose of identifying the beginning and end of the 5.9 second interval such that both singers and or listeners are able to employ the music for purposes of synchronizing their breathing to the audible cues and thereby achieving coherence of their respective heart rate variability cycles. The score of Silent Night 3A is used for this example. In this case, the tempo 3B is adjusted to allow markers to be inserted at 5.9 second intervals while in keeping with the basic tempo of the musical piece. In this example, chime 3C, 3D, 3E, 3F, 3G is inserted every 5.9 seconds 3H. The chime or equivalent musical cue can be integrated into the music at the time the music is being played or can be mixed in post recording. Of course, the degree to which the marker integrates pleasantly with the score has to do with the tempo of the music and the degree to which it conforms with the 5.9 second interval.

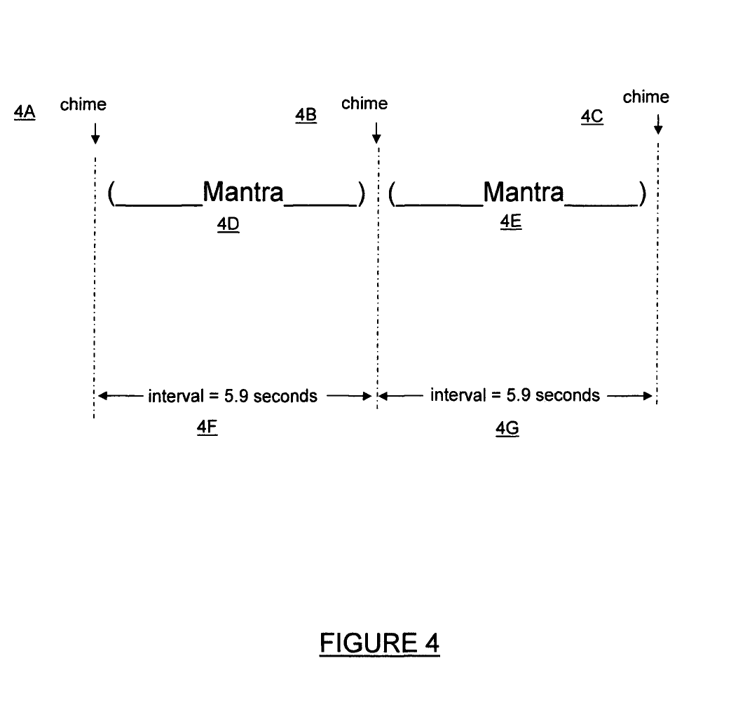

FIG. 4 depicts the application of the 5.9 second breathing interval to the repetition of “mantra”, mantra 4D, 4E, typically being a short verse or phrase of spiritual significance that is repeated over and over again. In this example, the 5.9 second interval 4F, 4G, is delineated by a chime 4A, 4B, 4C that occurs at the beginning of each 5.9 second interval. The mantra itself may fill this 5.9 second interval as well as span the interval with different verses.

Because both song and mantra are activities in which groups of people often participate, when music or mantra incorporating breathing cues is utilized the heart rate variability cycles of participants are synchronized. This is a nascent field of investigation about which much will be learned via the application of the present invention.

A discussion of visual presentation methods will now ensue. A similar requirement exists for visual indicators as for audio indicators, that is, it is desirable that the visual indicator communicate as much information as is possible about the 5.9 second cycle to the user. This includes changes from inhalation to exhalation and visa versa, whether the phase is an inhalation phase or an exhalation phase, and progression of the 5.9 second interval. Visual indicators may work alone or in combination with audible presentation methods previously described.

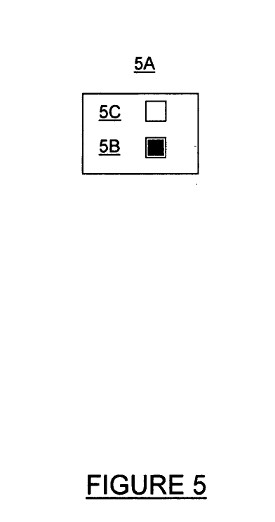

Referring to FIG. 5, the most basic method is represented by 5A which is a simple indication of change of breathing phase, that is, the change from inhalation to exhalation or visa versa. During the inhalation phase, indicator 5B is highlighted, during the exhalation phase indicator 5C is highlighted.

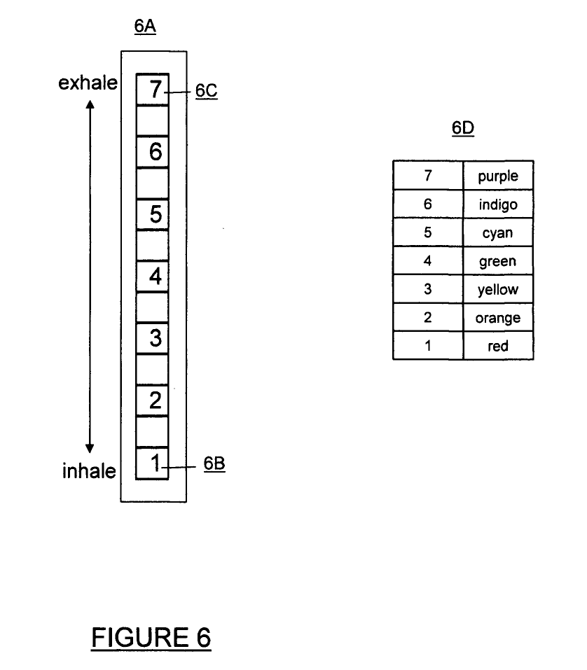

FIG. 6 presents a 13 segment indicator 6A that denotes the change of cycle, the present phase of the cycle, that is, inhalation or exhalation, and progression of the cycle. Inhalation is indicated by the transition from 1 to 7, exhalation is indicated by the transition from 7 to 1. It employs the 7 stage convention as did audio examples 2E and 2F. A second convention is applied to this visual display, this being the association of specific colors with specific numeric stages of progression. This association is depicted in the table 6D. Each segment of display 6A is illuminated for 1/13th of the fundamental 5.9 representative of both the end of the inhalation phase and the beginning of the exhalation phase, they are highlighted for 2 consecutive beats.

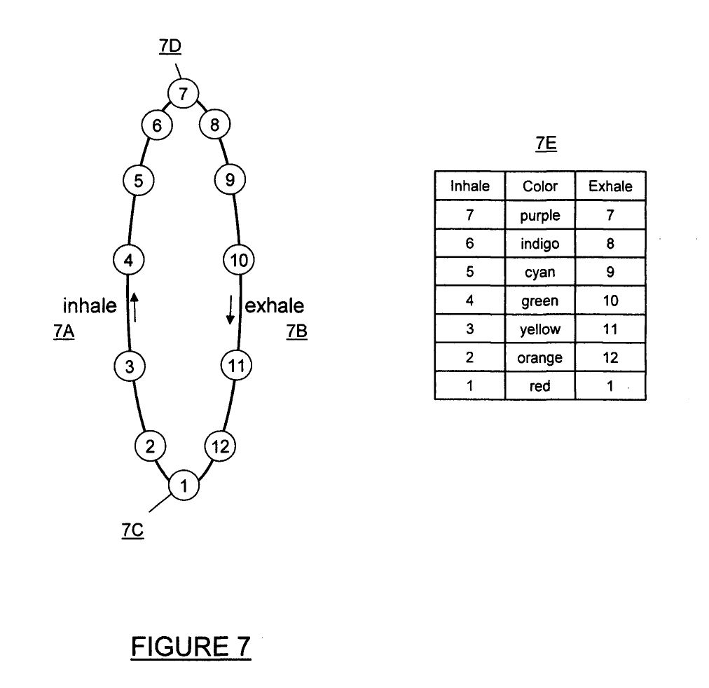

Referring to FIG. 7, these conventions and their relevance to breathing and heart rate variability will now be discussed. Preferred audible and visual embodiments of the present invention employ the 7 stage convention with inhalation occurring on increasing number and exhalation occurring on decreasing number. The 7 stage convention conforms to traditional yogic theory of energy planes or centers that exist in the body. When a subject employs the prescribed breathing method, that is synchronization of their breathing cycle with an external timing reference that is tuned to the natural heart rate variability cycle, the heart rate variability cycle will synchronize with the breathing cycle. When a subject practices this technique for a period of time, there is an internal perception that the energy in the body progresses to these different centers in a fashion consistent with the cycle of breath. That is, as inhalation occurs 7A, the energy moves from the lowermost center of the body, corresponding to the number 1, 7C, to the uppermost center of the body corresponding to the number 7, 7D via display segments 2, 3, 4, 5 and 6. As exhalation occurs 7B, the energy moves from the uppermost center of the body corresponding to the number 7, 7D, to the lowermost center of the body corresponding to the number 1, 7C via display segments 8, 9, 10, 11, and 12. As this begins to happen, this energy movement and its location in the body becomes an important dimension of the perceived progression of the breathing cycle. A consistent association of color with number is applied to this display according to table 7E. Relative to this specific display, there are 13 segments, 5 of which are paired.

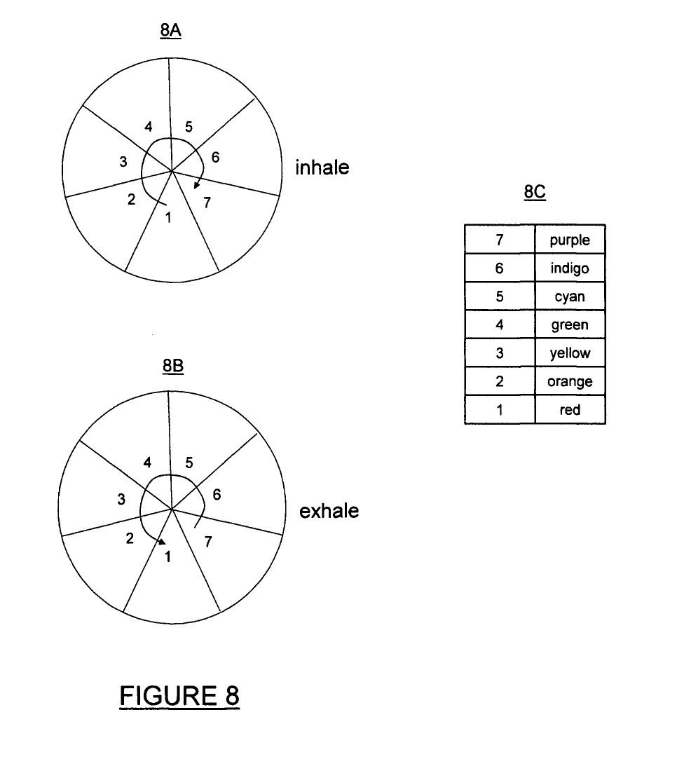

Referring to FIG. 8, 8A and 8B are simply circular representations of a single 7 segment indicator. Sectors are sequentially highlighted from 1 to 7 and 7 to 1. 8A demonstrates the inhalation phase of the cycle and 8B demonstrates the exhalation phase of the cycle. That is, as inhalation occurs 8A, the energy moves from the lowermost center of the body, corresponding to the number 1, to the uppermost center of the body corresponding to the number 7. Each sector is illuminated for 1/7th of the fundamental 5.9 second period or 0.84 seconds. A consistent association of color and number is applied to this display according to table 8C.

As in the case of music or mantra being engaged in simultaneously by multiple participants resulting in group synchronization of the heart rate variability cycle, the same applies to any form of the invention in which a group can participate including audible and visual forms. These might include school rooms, board rooms, concert halls, etc. This might also include wide area groups including local, regional, national, and global participation via television, internet, and radio broadcast. Again, as this is a nascent field of investigation much will be learned in this regard via the application of the present invention.

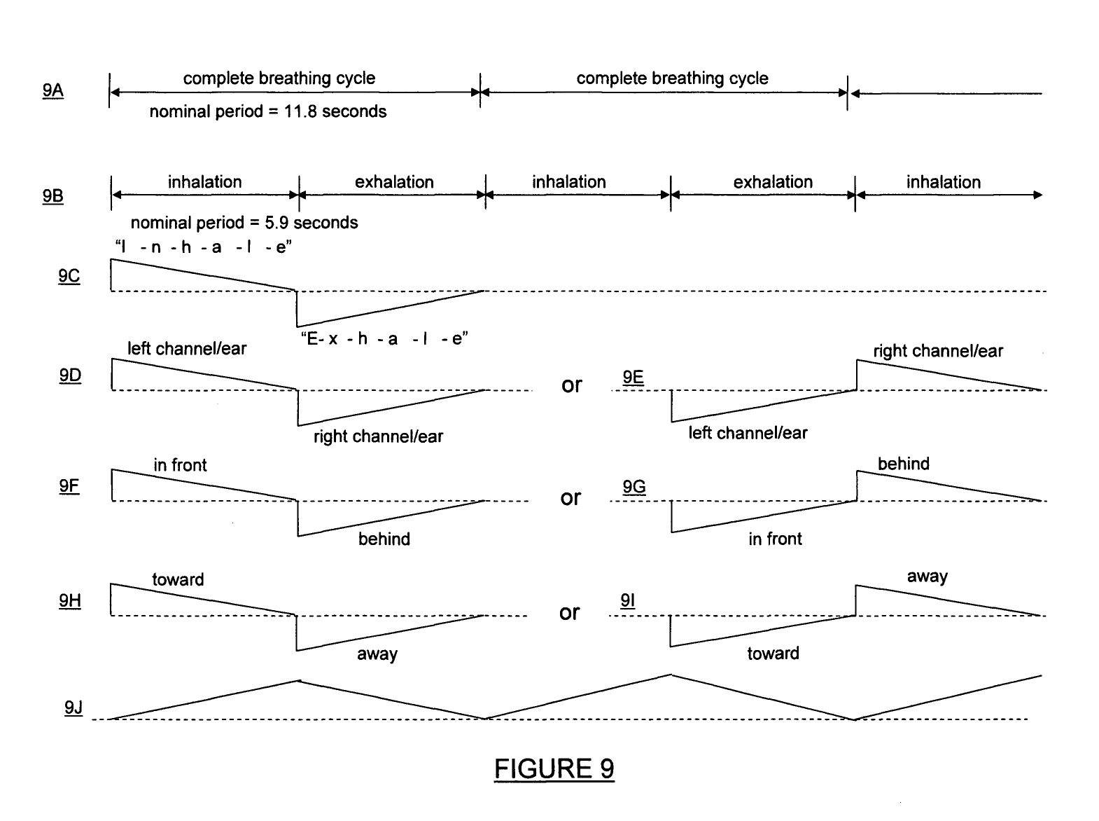

FIG. 9 presents fundamental audio methods that were not explicitly specified in prior patent Ser. No. 10/802,456. 9A and 9B present the basic breathing interval as per prior discussion. 9C represents the generic case wherein a recurring verbal word or command, in this case “inhale” and “exhale” signals the beginning of the inhalation or exhalation phase as well as communicates the progression of the interval. 9D and 9E represent the cases wherein the indication to inhale or exhale is made clear via a signal in either the right or left ear respectively. This method can work both ways, that is, the indication to inhale can be provided via the right ear or the left ear. The same is true for exhalation. The requirement is that the signal to inhale and the signal to exhale occur in opposite ears.

FIGS. 9F-9I present cases wherein psychoacoustic functions of human hearing are employed to distinguish between indications to inhale and exhale. 9F employs the audio engineering function “panning” to create the perception that the source of the sound is either in front of or behind the listener. Either “frontal” or “rear” positioning can be associated with either inhalation or exhalation. As per the discussion of 9D and 9E, the important requirement is that signals indicating inhalation and exhalation are opposite.

FIGS. 9H and 9I are representative of the psychoacoustic function wherein the sound appears to be moving “toward” and “away” from the listener, toward indicating when to inhale and away indicating when to exhale. Either toward or away can be associated with either inhalation or exhalation. As per 9F and 9G, the important requirement is that they be opposite.

FIG. 9J presents the very basic case wherein increasing and decreasing volume is employed to indicate both changes and progression of objective breathing phases.

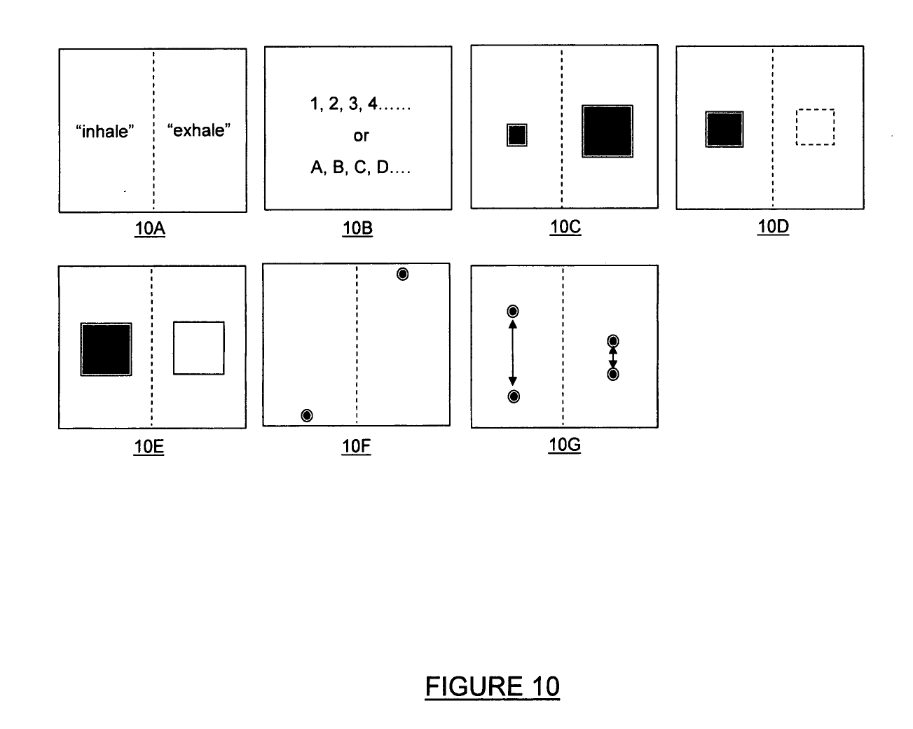

FIG. 10 presents fundamental visual methods not explicitly specified in prior patent Ser. No. 10/802,456. 10A specifies the use of a visually displayed word or command indicating when to inhale and exhale. In this case “inhale” and “exhale” are used as example. 10B specifies the use of a sequence of numbers or letters, either in natural or reverse order, to represent breathing phase. 10C specifies the use of a visual object or area that increases and decreases in size to indicate breathing phases. 10D specifies the use of an object that appears and disappears to indicate breathing phases. The appearance and disappearance is assumed to be gradual. In this regard it is a special case of 10C. 10E specifies an object or area that changes in color to indicate breathing phases. This color change can involve any or all color attributes of hue, saturation, or luminance. 10F employs the very basic case of a point or object moving vertically or horizontally, but not limited thereto, to indicate breathing phase. Inhalation is indicated by one direction and exhalation by the reverse. 10G employs a point or area that “vibrates” visually, changing in both frequency and amplitude, to indicate breathing phase and progression of the objective breathing cycle. As with other methods, the requirement is that inhalation and exhalation be represented by opposite visual phases.

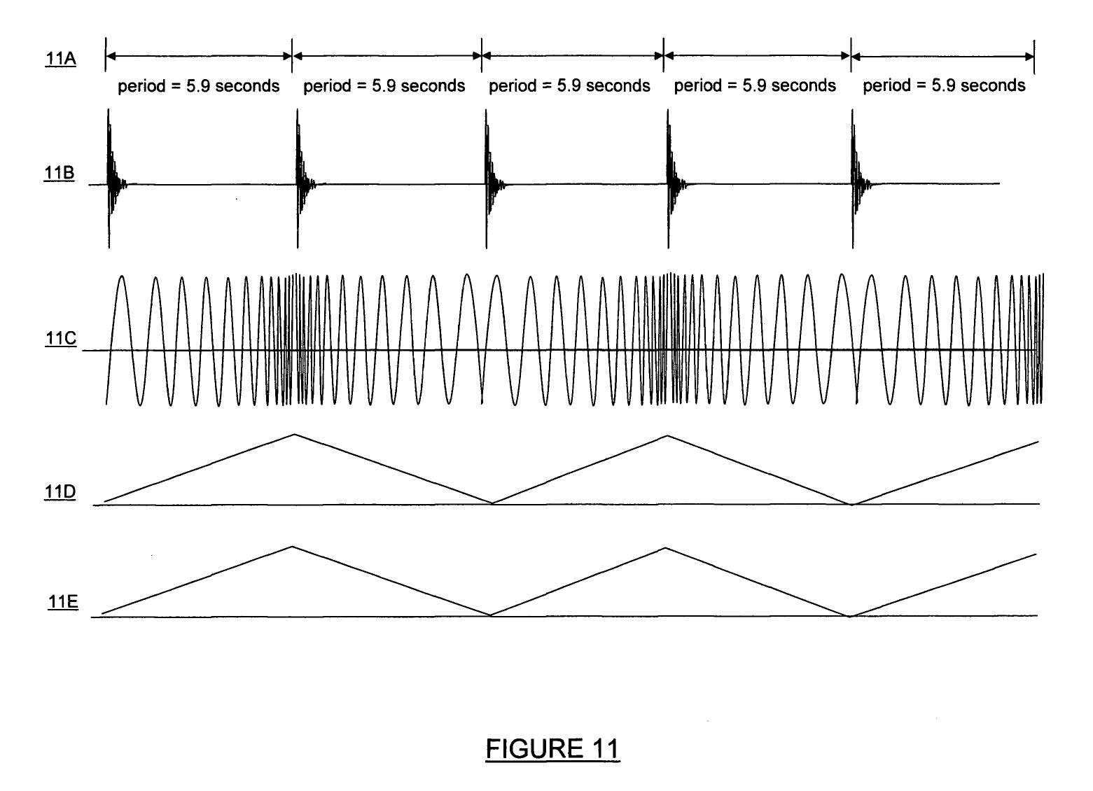

FIG. 11 specifies “sensory” methods other than audible or visual, specifically those having to do with touch, pressure, and motion. FIG. 11B presents the simple case wherein a short vibratory pulse is applied against the skin to indicate a change in breathing cycle. FIG. 11C presents the case wherein a vibration increases and decreases in rate as a function of the objective breathing cycle. FIG. 11D presents the case wherein pressure, as might be applied via a pressure cuff, is increased and decreased in accordance with the objective breathing cycle. FIG. 11E presents the case wherein a body part is moved in accordance with the objective breathing cycle, for example, a finger is raised and lowered. Relative to cases 11D, E, and F, inhalation or exhalation can be represented by either “activating” or “deactivating” phases.

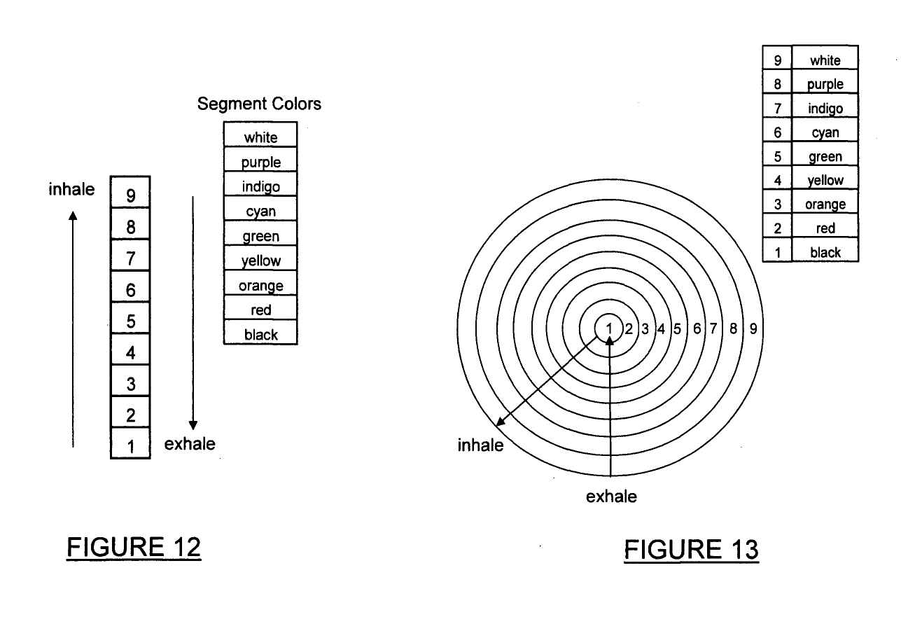

FIG. 12 presents a 9 segment linear display that may be presented vertically or horizontally. Colors are specified according to the table of FIG. 12. Illumination of segments 1 to 9 occurs sequentially coincident with inhalation and segments remain illuminated until the process is reversed coincident with exhalation.

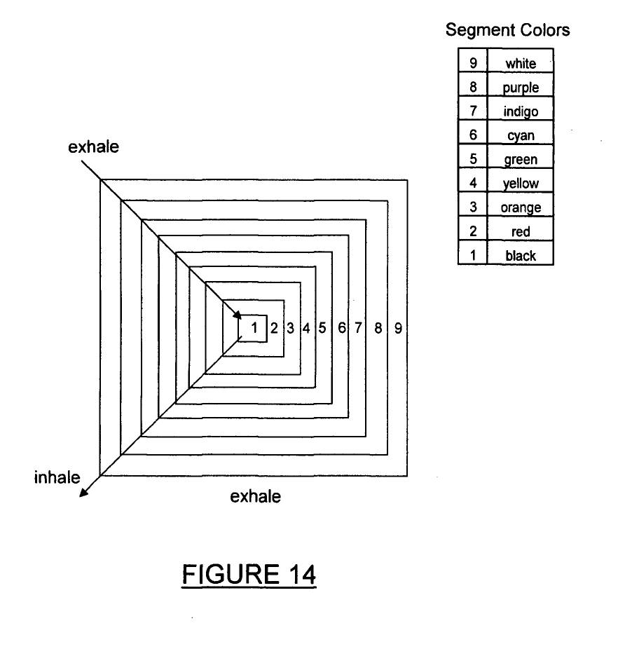

FIG. 13 presents a similar 9 segment circular display. Colors are specified according to the table of FIG. 13. Illumination of segments 1 to 9 occur sequentially from the center outward coincident with inhalation and remain illuminated until the process is reversed coincident with exhalation.

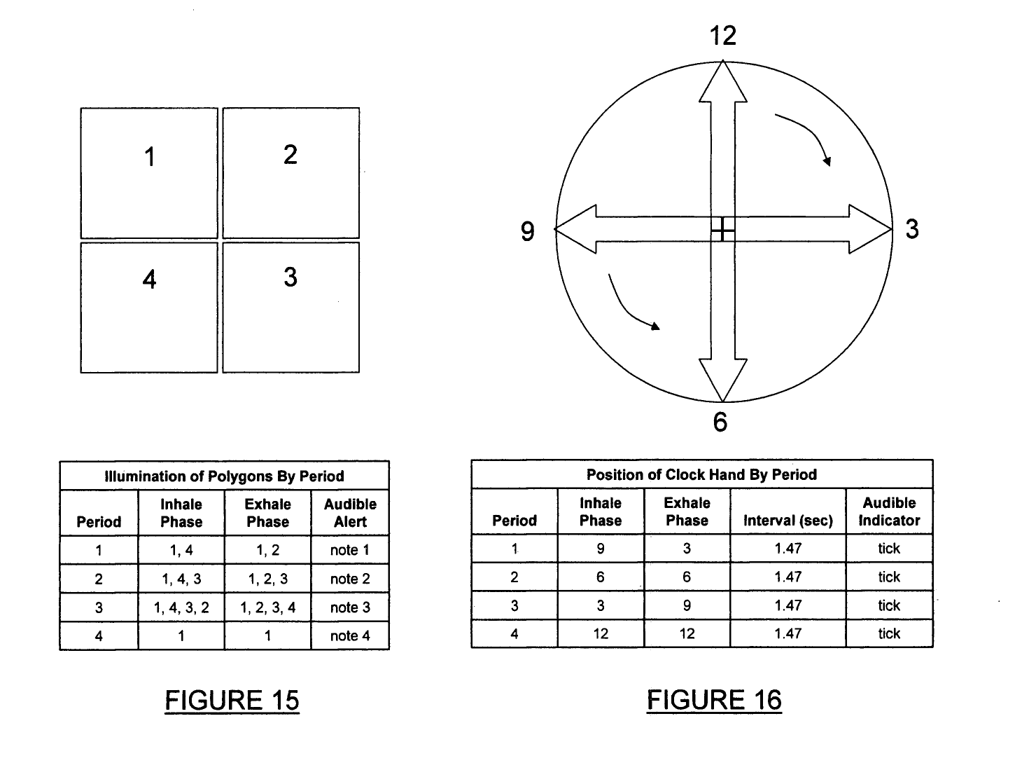

FIG. 14 presents a 9 segment regular polygonal display. A “square” is depicted by way of example of a regular polygon. The design in intended to apply to any and all regular polygons including the triangle, pentagon, hexagon, heptagon, octagon, nonagon, etc. Illumination of segments 1 to 9 occurs sequentially from the center outward coincident with inhalation and remain illuminated until the process is reversed coincident with exhalation.

FIG. 15 presents a simple 4 segment audiovisual display consisting of 4 regular polygons arranged and displayed in a geometrically contiguous fashion such that exhalation is aligned with the illumination of polygons in the clockwise order 2, 3, 4, 1, and inhalation is aligned with the illumination of polygons in the counterclockwise order 4, 3, 2, 1 order, a musical note occurring with each transition. Polygon 1 is illuminated at all times. Polygons 2, 3, and 4 are extinguished each time period 4 is asserted.

Similarly, FIG. 16 presents a clock face, the hand of which moves to indicate the progression of the breathing interval, each “tick” indicating 25% of the interval or 1.47 seconds, the hand moving in the clockwise direction and pausing at 3, 6, 9, and 12 on exhalation, and the hand moving counterclockwise pausing at 9, 6, 3, and 12 on inhalation. An audible “tick” occurs with each movement of the clock hand.

1. The broad method of presenting audible and visual cues for synchronizing the breathing cycle with an external timing reference for purposes of synchronizing the heart rate variability cycle with the breathing cycle, thereby achieving coherence of the heart rate variability cycle:
a) The broad method of using audible indicators to communicate inhalation and exhalation phases of breathing, changes of said phases, progression of said phases in time, and progression of said phases relative to the internal perception of the practitioner.
b) The broad method of using visual indicators to communicate inhalation and exhalation phases of breathing, changes of said phases, progression of said phases in time, and progression of said phases relative to the internal perception of the practitioner.
c) The broad method of using sensory methods other than audible or visual methods to communicate inhalation and exhalation phases of breathing, changes of said phases, and progression of said phases in time.

2. The method of claim 1 wherein audible, visual, or sensory indicators are used individually or in combination to accurately indicate objective breathing phases for purposes of synchronizing the breathing cycle with an external timing reference.

3. The method of claim 1 wherein increasing and decreasing volume is used as the basis of indicating breathing phase for purpose of synchronizing the breathing cycle with an external timing reference.

4. The method of claim 1 wherein recurring recitation of verbal words or commands are employed either audibly or visually to indicate changes in breathing phase.

5. The method of claim 1 wherein the recurring recitation of numbers in either natural or reverse order is employed either audibly or visually to indicate changes and progression of the objective breathing cycle.

6. The method of claim 1 wherein a recurring change of ears or “channels” is employed to distinguish between inhalation and exhalation phases of the objective breathing cycle.

7. The method of claim 1 wherein changes in the psychoacoustic perception of “space” and “motion” are employed to indicate inhalation and exhalation phases as well as progression of the objective breathing cycle.

8. The method of claim 1 wherein the recurring visual appearance and disappearance of an object, and the rate of said appearance or disappearance is employed to indicate both phases and progression of the objective breathing cycle.

9. The method of claim 1 wherein a recurring change in the visual size or shape of an object is employed to indicted breathing phases and progression of said breathing phases of the objective breathing cycle.

10. The method of claim 1 wherein the movement of a point or area, either up and down, left and right, or any combination and permutation of up and down, left and right, is employed to indicate phases and progression of said phases of the objective breathing cycle.

11. The method of claim 1 wherein the motion of a point or object changes in frequency and amplitude is employed to indicate breathing phases and progression of said phases of the objective breathing cycle.

12. The method of claim 1 wherein a simple vibratory pulse is applied against the skin to indicate the basic change of breathing cycle.

13. The method of claim 1 wherein a recurring increase and decrease in vibration is employed to indicate breathing phases and progression of said phases of the objective breathing cycle.

14. The method of claim 1 wherein increasing and decreasing pressure, for example as applied via a pressure cuff, is varied in accordance with the phases and progression of the objective breathing cycle.

15. The method of claim 1 wherein the recurring movement/motion of a body part, for example the mechanical raising and lowering the finger, is employed to indicate breathing phases and progression of said phases of the objective breathing cycle.

16. The method of claim 1 wherein 9 segment linear, circular, and polygonal visual displays are illuminated from 1 to 9 representing inhalation, segments remaining illuminated until the process is reversed upon the exhalation phase. Specific colors are 1=black, 2=red, 3=orange, 4=yellow, 5=green, 6=cyan, 7=indigo, 8=purple, 9=white.

17. The method of claim 1 wherein 9 musical notes are employed in melodic sequence to represent the inhalation or exhalation phase of the breathing cycle.

18. The audio visual method wherein 4 regular polygons are illuminated and extinguished in a geometrically contiguous fashion, the display of each indicating the elapsing of 25% or 1.47 seconds of the total interval, along with the coincident occurrence of a distinct note associated with the appearance or disappearance of each polygon.

19. The audio visual method wherein the movement of the hand of a clock occurs in a clockwise direction beginning at 12 and stopping at 3, 6, 9, and 12, and returns in a counterclockwise direction stopping at 9, 6, 3, and 12, each position indicating the elapsing of 25% or 1.47 seconds of the total interval, along with the audio indication of a clock “tick” occurring coincident with each movement of the clock hand.

20. The instantiation of specified audiovisual methods in hardware optimized, software optimized, and mechanical instruments.
